(19) United States
(12) Patent Application Publication
(10) Pub. No.: US 2014/0030360 A1
(43) Pub. Date: Jan. 30, 2014

(54) CHLORINE DIOXIDE GEL AND MANUFACTURING METHOD THEREOF

(71) Applicant: Hao Chang Yin, New Taipei City (TW)

(72) Inventors: Hao Chang Yin, New Taipei City (TW); Shan Shue Wang, New Taipei City (TW); Allen Bai, New Taipei City (TW)

(73) Assignee: Hao Chang Yin, New Taipei City (TW)

(21) Appl. No.: 13/948,175

(22) Filed: Jul. 23, 2013

(30) Foreign Application Priority Data
Jul. 24, 2012 (TW) 101126583

(51) Int. Cl. A61K 47/10

(52) U.S. Cl. CPC A61K 47/10
USPC 424/661

(57) ABSTRACT

A method for manufacturing chlorine dioxide gel includes the following steps: a step of dissolving peg-copolymer bis-decyltetradeceth in hot water to form an aqueous solution of peg-copolymer bis-decyltetradeceth; a step of heating the aqueous solution of peg-copolymer bis-decyltetradeceth obtained in the above step to form a gel like colloid; a step of dissolving pure chlorine dioxide gas in water to form an aqueous solution of chlorine dioxide; and a step of uniformly mixing the aqueous solution of chlorine dioxide and the colloid of peg-copolymer bis-decyltetradeceth to form a chlorine dioxide gel, whereby with the above steps, a method for manufacturing chlorine dioxide gel that stably preserves chlorine dioxide gas in a gel is provided.

### TECHNICAL FIELD OF THE INVENTION

The present invention generally relates to a method for manufacturing a chlorine dioxide gel, and more particularly to a method for manufacturing a chlorine dioxide gel that stably preserves chlorine dioxide gas in a gel.

### DESCRIPTION OF THE PRIOR ART

Chlorine dioxide (ClO₂) has an unpaired free electron so that chlorine dioxide has extremely effective oxidizing capability and is used to oxidize for example protein, peptide, DNA, or RNA of pathogens, such as bacteria, viruses, and fungus for terminating the pathogens. Nowadays, chlorine dioxide has replaced chlorine gas or ozone for wide use in disinfection, sterilization, and deodorization in the industries of medical hygiene, food processing, environmental protection, industrial water, husbandry and cultivation, and sewage treatment.

However, since chlorine dioxide gas is a powerful oxidizer, the preservation thereof is difficult. Chlorine dioxide gas may continuously release and dissipate even if it is dissolved in water. To long preserve chlorine dioxide gas for subsequent use, there are three ways that are conventionally adopted. The first way is to have chlorine dioxide absorbed in the solution of sodium carbonate (Na₂O₃), sodium borate (Na₂B₄O₇ 10H₂O), hydrogen peroxide (H₂O₂), or sodium hydroxide (NaOH) to allow chlorine dioxide to convert into the form of chlorite (ClO₂). Before use, the solution must be activated or acidified to generate gaseous chlorine dioxide. Another way is to use two agents of chlorite and acid and before use, the two agents are added to water of a proper amount to allow chlorite and the acid to react with each other for generating chlorine dioxide solution for subsequently providing chlorine dioxide gas. A further way is to prepare chlorine dioxide gas through electrolysis, in which saturated salt brine is used as the electrolyte so that after electricity is supplied between the anode and the cathode, chlorine dioxide gas is generated at the anode.

Although it is convenient to use chemical acid process or electrolysis to prepare chlorine dioxide, it is required that before, the agents must mixed or chlorite solution must be activated before use or saturated salt brine must be electrolyze in order to generate chlorine dioxide gas. The operation is complicated and inconvenience and is disadvantageous in that it cannot fast obtain chlorine dioxide gas. Further, whether chlorine dioxide gas is obtained through chemical acid process or electrolysis, it will be continuously release and dissipate in the atmosphere and the concentration of the chlorine dioxide gets lowering with the lapse of time. This leads to a disadvantage that long preservation for subsequent disinfection and sterilization is generally impossible. Thus, long preservation of chlorine dioxide gas is generally an issue to be addressed.

### SUMMARY OF THE INVENTION

An object of the present invention is to provide a method for manufacturing chlorine dioxide that uses a gel to preserve chlorine dioxide gas so as to achieve long preservation of chlorine dioxide gas.

To achieve the above object, the present invention provides a method for manufacturing a chlorine dioxide gel, which comprises the following steps:

Step 1: dissolving peg-copolymer bis-decyltetradeceth in hot water to form an aqueous solution of peg-copolymer bis-decyltetradeceth;

Step 2: heating the aqueous solution of peg-copolymer bis-decyltetradeceth obtained in the above step to form a gel like colloid;

Step 3: dissolving pure chlorine dioxide gas in water to form an aqueous solution of chlorine dioxide; and

Step 4: uniformly mixing the aqueous solution of chlorine dioxide and the colloid of peg-copolymer bis-decyltetradeceth of Step 2 to form a chlorine dioxide gel.

These steps provide a method for manufacturing chlorine dioxide gel that stably preserves chlorine dioxide gas in a gel.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts. Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a flow chart showing a manufacturing method according to the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

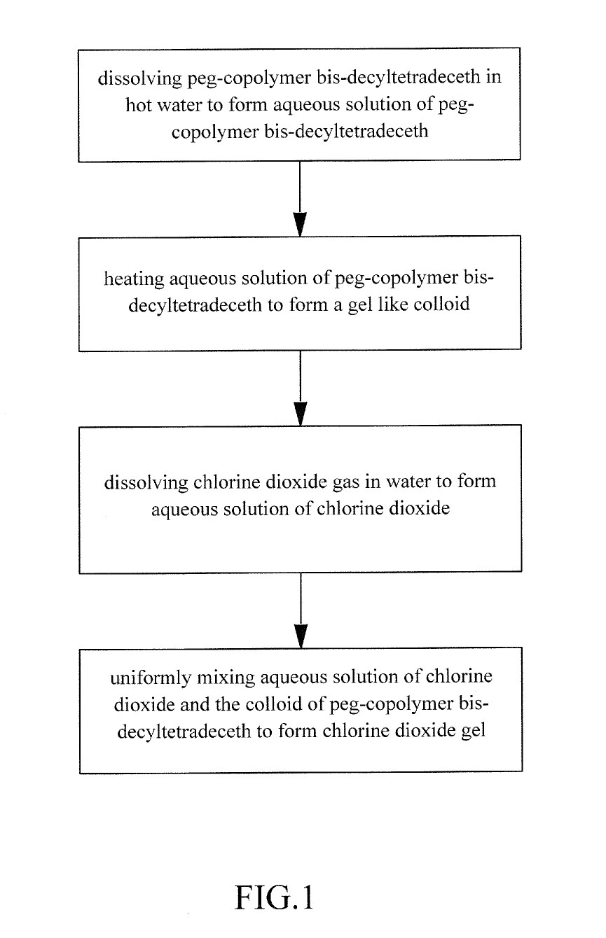

Referring to the flow chart shown in FIG. 1, the present invention provides a method for manufacturing chlorine dioxide gel, which comprises the following steps:

Step 1: dissolving peg-copolymer bis-decyltetradeceth in hot water to form an aqueous solution of peg-copolymer bis-decyltetradeceth, wherein peg-copolymer bis-decyltetradeceth of 50 grams is dissolved in 1,000 cc hot water having a temperature of 90° C. to form the aqueous solution of peg-copolymer bis-decyltetradeceth;

Step 2: heating the aqueous solution of peg-copolymer bis-decyltetradeceth obtained in the above step to form a gel like colloid, wherein the aqueous solution of peg-copolymer bis-decyltetradeceth is heated to 90° C. to allow the aqueous solution of peg-copolymer bis-decyltetradeceth to form a gel-like colloid;

Step 3: dissolving pure chlorine dioxide gas in water to form an aqueous solution of chlorine dioxide; and

Step 4: uniformly mixing the aqueous solution of chlorine dioxide and the colloid of peg-copolymer bis-decyltetradeceth to form a chlorine dioxide gel, wherein the colloid of peg-copolymer bis-decyltetradeceth that has been cooled down to 30° C. is uniformly mixed with the aqueous solution of chlorine dioxide to form the chlorine dioxide gel.

With the above steps, peg-copolymer bis-decyltetradeceth is first dissolved and heated to form a gel-like colloid and is then uniformly mixed with the aqueous solution of chlorine dioxide. With the property and characteristics of the colloid of peg-copolymer bis-decyltetradeceth and the feature that the colloid of peg-copolymer bis-decyltetradeceth does not cause oxidization or reduction reaction with the aqueous solution of chlorine dioxide, chlorine dioxide gas can be stably stored in the colloid of peg-copolymer bis-decyltetradeceth and chlorine dioxide is prevented from releasing and dissipating in the atmosphere so as to form a chlorine dioxide gel that can stably preserve chlorine dioxide gas.

The chlorine dioxide gel manufactured with the above steps contains therein a large quantity of chlorine dioxide gas. The gel can be applied to a wound for use of the strong oxidation capability of chlorine dioxide to oxidize and decompose amino acids of bacteria and viruses in the wound for realizing disinfection and sterilization. Further, with the chlorine dioxide gel applied to the surface of the wound, a protection layer can be formed to isolate the wound from the surrounding atmosphere so as to reduce the occurrence of infection. Further, besides long and effectively preserving chlorine dioxide, using the colloid of peg-copolymer bis-decyltetradeceth to form a chlorine dioxide gel for preservation chlorine dioxide gas allows chlorine dioxide gas preserved in the gel to maintain a predetermined concentration and also allows a user to efficiently access and use the gel without spending a lot of time to waiting for the completion of preparation of chlorine dioxide so that advantages of easy and efficient use and disinfection and sterilization can be achieved.

Further, since chlorine dioxide gas has a powerful capability of oxidization, applying the gel to buildings, fruits, or seeds can use chlorine dioxide gas to suppress the growth of fungus and mildew and also effectively preventing the formation of protection layer thereby extending the preservation period of the fruits or seeds and enhancing convenience of shipping.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A method for manufacturing chlorine dioxide gel, comprising the following steps:
(1) dissolving peg-copolymer bis-decyltetradeceth in hot water to form an aqueous solution of peg-copolymer bis-decyltetradeceth;
(2) heating the aqueous solution of peg-copolymer bis-decyltetradeceth obtained in the above step to form a gel like colloid;
(3) dissolving pure chlorine dioxide gas in water to form an aqueous solution of chlorine dioxide; and
(4) uniformly mixing the aqueous solution of chlorine dioxide and the colloid of peg-copolymer bis-decyltetradeceth of step (2) to form a chlorine dioxide gel,
whereby with the above steps, a method for manufacturing chlorine dioxide gel that stably preserves chlorine dioxide gas in a gel is provided.
